(19) United States
(12) Patent Application Publication
(10) Pub. No.: US 2024/0108464 A1
(43) Pub. Date: Apr. 4, 2024

(54) EXPANDABLE PROSTHETIC HEART VALVE FRAME WITH LEAFLET SUPPORT STRUTS

(71) Applicant: EDWARDS LIFESCIENCES CORPORATION, Irvine, CA (US)

(72) Inventors: Tamir S. Levi, Zikhron Yaakov (IL); Nikolai Gurovich, Hadera (IL)

(21) Appl. No.: 18/536,823

(22) Filed: Dec. 12, 2023

Related U.S. Application Data
(63) Continuation of application No. PCT/US2022/033288, filed on Jun. 13, 2022.
(60) Provisional application No. 63/210,448, filed on Jun. 14, 2021.

(51) Int. Cl. A61F 2/24

(52) U.S. Cl. CPC A61F 2/2418; A61F 2210/0057; A61F 2220/0075

(57) ABSTRACT

A prosthetic heart valve including a radially expandable and compressible annular frame and a plurality of leaflets attached to the frame is disclosed. The frame includes a plurality of interconnected struts defining a plurality of rows of cells arranged between a first end and a second end of the frame, the plurality of interconnected struts comprising a plurality of first struts defining the first end of the frame. The frame further includes a plurality of second struts axially offset from the first end of the frame, each second strut connected to and between two adjacent first struts of the plurality of first struts and extending axially away from the two adjacent first struts, where an inflow end of a cusp edge portion of each leaflet of the plurality of leaflets is secured to a corresponding second strut of the plurality of second struts.

### CROSS-REFERENCE TO RELATED APPLICATIONS

This application is a continuation of PCT Application No. PCT/US2022/033288, filed Jun. 13, 2022, which claims the benefit of U.S. Provisional Patent Application No. 63/210,448, filed Jun. 14, 2021, all of which are incorporated by reference herein in their entireties.

### FIELD

The present disclosure relates to expandable prosthetic heart valves including a frame with support struts configured to support cusp edge portions of leaflets of the expandable heart valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery device so that the prosthetic valve can self-expand to its functional size.

Most expandable, transcatheter heart valves comprise a radially expandable and compressible cylindrical metal frame and prosthetic leaflets mounted inside the frame. The frame can comprise a plurality of circumferentially extending rows of angled struts defining rows of open cells of the frame.

Prosthetic heart valve design can affect both short- and long-term durability of the prosthetic heart valve, as well as serviceability when subsequent intervention is required. An important design parameter of prosthetic heart valves is the amount of mechanical interaction between the frame of the prosthetic heart valve and the native anatomy at the annulus and left ventricle outflow tract (LVOT) level. For example, for a prosthetic aortic valve, an inflow end of the frame can extend into the LVOT and, in some examples, interact with the left ventricular wall at the LVOT level and result in conductance disturbances.

Further, interactions between components of the prosthetic heart valve, such as a skirt disposed around an outer surface of the frame, and the surrounding tissue may trigger an adverse tissue response.

Accordingly, a need exists for improved frame and skirt designs for prosthetic heart valves.

### SUMMARY

Described herein are examples of prosthetic heart valves including a radially expandable and compressible annular frame comprising a plurality of interconnected struts. The prosthetic heart valve can further include a leaflet assembly comprising a plurality of leaflets secured to the frame. The struts of the frame can define a plurality of rows of cells arranged between an inflow end and an outflow end of the frame. In some examples, the struts can include a plurality of first struts that define the inflow end of the frame. The prosthetic heart valve can include a plurality of second struts that are axially offset from the inflow end of the frame, where each second strut is connected to and between two adjacent first struts and extends axially away from the two adjacent first struts. In some examples, an inflow end of a cusp edge portion of each leaflet can be secured to one of the second struts such that the inflow end is axially offset from the inflow end of the frame.

In one representative example, a prosthetic heart valve comprises a radially expandable and compressible annular frame, the frame comprising: a plurality of interconnected struts defining a plurality of rows of cells arranged between a first end and a second end of the frame, the plurality of interconnected struts comprising a plurality of first struts defining the first end of the frame. The frame further comprises a plurality of second struts axially offset from the first end of the frame, each second strut connected to and between two adjacent first struts of the plurality of first struts and extending axially away from the two adjacent first struts. The prosthetic heart valve further comprises a plurality of leaflets attached to the frame, each leaflet of the plurality of leaflets comprises a cusp edge portion and a free edge portion, and an inflow end of the cusp edge portion of each leaflet of the plurality of leaflets is secured to a corresponding second strut of the plurality of second struts such that the inflow end is axially offset from the first end of the frame.

In another representative example, a prosthetic heart valve comprises: a radially expandable and compressible annular frame. The frame comprises a plurality of interconnected struts defining a plurality of rows of cells arranged between an inflow end and an outflow end of the frame, the plurality of interconnected struts comprising: a plurality of first struts defining a plurality of commissure windows disposed adjacent to the outflow end of the frame; and a plurality of second struts arranged end-to-end and extending between the plurality of commis sure windows and the inflow end of the frame in an undulating pattern around a circumference of the frame. The frame further comprises a plurality of third struts axially offset from the inflow end of the frame, each third strut of the plurality of third struts connected to two adjacent second struts of the plurality of second struts which form a part of the inflow end of the frame.

In another representative example, a prosthetic heart valve comprises: a radially expandable and compressible annular frame comprising: a plurality of interconnected struts defining a plurality of rows of cells arranged between an inflow end and an outflow end of the frame and configured to support a radial expansion and compression of the frame, the plurality of interconnected struts including a plurality of first struts arranged end-to-end around a circumference of the frame and forming the inflow end of the frame. The frame further comprises a plurality of apices formed at the inflow end of the frame, each apex of the plurality of apices formed at a junction between two adjacent first struts of the plurality of first struts. The frame further comprises a plurality of arcuate second struts that extend axially beyond the inflow end of the frame, where each second strut of the plurality of second struts is connected to and between two adjacent apices of the plurality of apices and is configured to bend at a central region of the second strut during radial compression of the frame.

In another representative example, a prosthetic heart valve comprises: a radially expandable and compressible annular frame comprising a plurality of interconnected struts; a plurality of leaflets arranged on an inside of the frame, each leaflet of the plurality of leaflets comprising two commissure tabs disposed on opposite sides of the leaflet and a cusp edge portion extending between the two commissure tabs, the cusp edge portions of the plurality of leaflets are attached directly to a plurality of first struts of the plurality of interconnected struts, and the plurality of first struts have a curved shape configured to follow a curvature of the cusp edge portions of the plurality of leaflets; and an outer skirt disposed around an outer surface of the frame.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2 is a side view of an example of a delivery apparatus configured to deliver and implant a radially expandable prosthetic heart valve at an implantation site.

FIG. 3 is a perspective view of a prosthetic heart valve, according to another example.

FIG. 4 shows an example of a frame for a prosthetic heart valve in a flattened configuration, the frame including a plurality of inflow support struts that are axially offset from an inflow end of the frame and are configured to support inflow ends of a plurality of leaflets of the prosthetic heart valve.

FIG. 5 shows a portion of a frame including an inflow support strut, according to an example, that has a narrowed width configured to increase a flexibility of the inflow support strut such that it does not interfere with the radial expansion and compression of the frame.

FIG. 6A show a portion of a frame including an inflow support strut, according to another example, that has one or more thinned regions configured to increase a flexibility of the inflow support strut such that it does not interfere with the radial expansion and compression of the frame.

FIG. 6B shows a detail portion of a thinned region of the inflow support strut of FIG. 6A.

FIG. 7 is a side view of a portion of a prosthetic heart valve comprising the frame of FIG. 4, leaflets attached directly to the frame, and an outer skirt disposed around an outer surface of the frame.

FIG. 8 is a side view of a portion of a prosthetic heart valve having an outer skirt, according to another example, which is configured to be arranged around an outer surface of the frame.

FIG. 9 is a side view of the portion of the prosthetic heart valve of FIG. 8 showing the outer skirt attached to the frame with a plurality of loose stitches.

FIG. 10 is a side view of a portion of a prosthetic heart valve having a frame and an outer skirt, according to another example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Examples of the Disclosed Technology

Described herein are examples of radially expandable and compressible prosthetic heart valves including an annular frame. The prosthetic heart valve may further include a plurality of leaflets attached to the frame. In some examples, the leaflets can be attached to the frame via commissures formed by joining pairs of adjacent ends (e.g., commissure tabs) of the leaflets.

In some examples, the frame of the prosthetic heart valve can include a plurality of rows of cells formed by interconnected struts of the frame. The plurality of rows of cells can be formed between an inflow end and an outflow end of the frame.

In some examples, the frame can additionally include a plurality of inflow support struts that are attached to junctions of the interconnected struts at the inflow end of the frame and that extend axially away from the inflow end of the frame. Each inflow support strut can be configured to support an inflow end of a cusp edge portion of a corresponding leaflet that is secured directly to the inflow support strut. In some examples, the inflow support struts can be curved and follow (or match) a curvature of the inflow end of the cusp edge portion of the leaflet. Thus, the inflow ends of the leaflets can be axially offset from the inflow end of the frame. As a result, outflow edges of the leaflets can be offset away from the outflow end of the frame, thereby creating open space within a first row of cells disposed at the outflow end of the frame. This can provide increased space for coronary access and re-access devices, as described herein.

In some examples, a first portion of the interconnected struts of the frame that extend from commissure windows of the frame to the inflow support struts can be curved such that they follow a curvature of the cusp edge portions of the leaflets. The cusp edge portions of the leaflets can then be attached directly to the first portion of the interconnected struts.

Further, in some examples, an outer skirt can be disposed around an outer surface of the frame and the leaflets can be arranged on an inside of the frame and secured directly to the struts of the frame (as described above) without being attached to an inner skirt.

In some examples, one or more of first and second ends (e.g., inflow and outflow ends) of the outer skirt can have an undulating shape that follows a profile of the struts of the frame. In some examples, the outer skirt is attached to the outer surface of the frame via a plurality of loose (or floating) stitches. Thus, stretching of the outer skirt in a longitudinal direction during compression of the frame can be reduced or avoided.

Interactions between components of the prosthetic heart valve, such as a skirt disposed around an outer surface of the frame, and the surrounding tissue may trigger an adverse tissue response that can result in thrombus formation in the short term and/or pannus formation in the long term. Thus, a location and composition of the materials included in the prosthetic heart valve can be important for the long-term durability of the prosthetic heart valve when implanted in vivo.

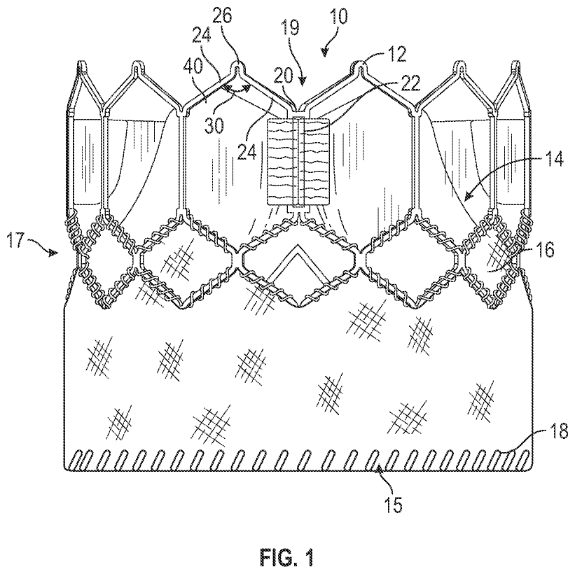

FIG. 1 shows a prosthetic heart valve 10, according to one example. The prosthetic heart valve disclosed herein can be implanted at various locations within a patient's vasculature. For example, any of the prosthetic valves disclosed herein can be implanted in the native aortic annulus. In other examples, the disclosed prosthetic heart valves can be implanted in the other native annuluses of the heart (e.g., the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve).

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

The valvular structure 14 can comprise three leaflets 40, collectively forming a leaflet structure, which can be arranged to collapse in a tricuspid arrangement, although in other examples there can be greater or fewer number of leaflets (e.g., one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

The frame 12 can be radially compressible (collapsible) and expandable (e.g., expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (e.g., stainless steel, etc.) or self-expanding materials (e.g., nickel titanium alloy (NiTi), such as nitinol), as known in the art. When constructed of a plastically-expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, a biocompatible, high-strength alloys (e.g., a cobalt-chromium or a nickel-cobalt-chromium alloys), polymers, or combinations thereof. In particular examples, frame 12 is made of a nickel-cobalt-chromium-molybdenum alloy, such as MP35N® alloy (SPS Technologies, Jenkintown, Pennsylvania), which is equivalent to UNS R30035 alloy (covered by ASTM F562-02). MP35N® alloy/UNS R30035 alloy comprises 35% nickel, 35% cobalt, 20% chromium, and 10% molybdenum, by weight. Additional details regarding the prosthetic valve 10 and its various components are described in WIPO Patent Application Publication No. WO 2018/222799, which is incorporated herein by reference.

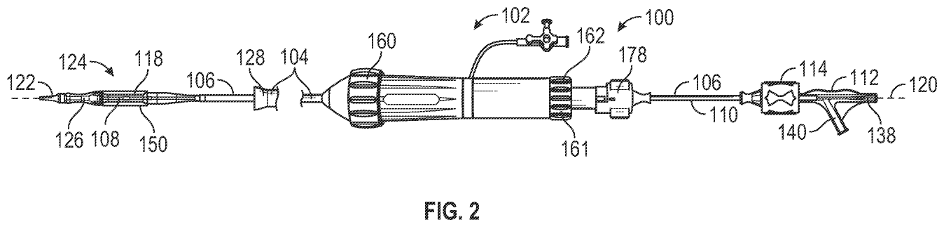

FIG. 2 shows a delivery apparatus 100, according to an example, that can be used to implant an expandable prosthetic heart valve (e.g., the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (e.g., move) longitudinally, along a central longitudinal axis 120 of the delivery apparatus 100, relative to one another to facilitate delivery and positioning of a prosthetic valve at an implantation site in a patient's body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (e.g., inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (e.g., a distal shoulder). An intermediate portion of the balloon 118 can overlay a valve mounting portion 124 of a distal end portion of the delivery apparatus 100 and a distal end portion of the balloon 118 can overly a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient's vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Pat. No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (e.g., for fine positioning at the implantation site). Further details on the delivery apparatus 100 can be found in U.S. Provisional Application Nos. 63/069,567 and 63/138,890, which are incorporated by reference herein.

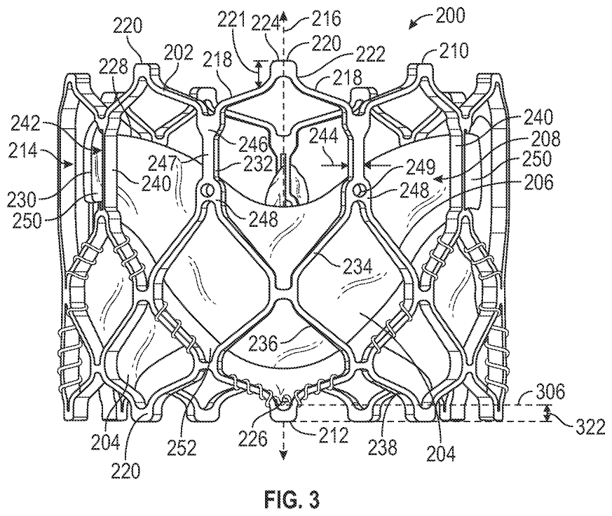

FIG. 3 shows an example of a prosthetic heart valve 200 comprising a radially expandable and compressible annular frame 202 and a plurality of leaflets 204 secured to the frame. Each leaflet 204 can comprise opposing commissure tabs 250 disposed on opposite sides of the leaflet 204 and a cusp edge portion 252 extending between the opposing commissure tabs. As described further below, the cusp edge portion 252 of each leaflet 204 can have an inflow end 226 (or edge, disposed at a central portion of the cusp edge portion 252) that is arranged at an inflow end of the prosthetic heart valve (e.g., inflow end 212 of a frame 202 of the prosthetic heart valve 200)

The frame 202 can be made of any of various suitable plastically-expandable materials (e.g., stainless steel, etc.) or self-expanding materials (e.g., nickel titanium alloy (NiTi or nitinol). In some examples, the frame 202 comprises a plastically-expandable material, such as those described above with reference to the prosthetic heart valve 10 of FIG. 1.

The frame 202 can comprise a plurality of interconnected struts 206 which form multiple rows of open cells 208 between an outflow end 210 and an inflow end 212 of the frame 202. In some examples, as shown in FIG. 3, the frame 202 can comprise three rows of cells 208 with a first (e.g., upper in FIG. 3) row of cells 214, disposed at the outflow end 210, having cells 208 that are elongated in an axial direction (relative to a central longitudinal axis 216 of the frame 202), as compared to cells 208 in the remaining rows of cells. For example, the cells 208 of the first row of cells 214 can have a longer axial length, defined in a direction of a central longitudinal axis 216 of the frame 202, than cells 208 in the remaining rows of cells (e.g., cells in the row of cells at the inflow end 212).

In some examples, as shown in FIG. 3, each row of cells 208 comprises nine cells. Thus, in such examples, the frame 202 can be referred to as a nine-cell frame.

In other examples, the frame 202 can comprise more than three rows of cells (e.g., four or five) and/or more or less than nine cells per row. In some examples, the cells 208 in the first row of cells 214 may not be elongated compared to cells 208 in remaining rows of cells of the frame 202.

The interconnected struts 206 can include a plurality of angled struts 218, 234, 236, and 238 arranged in a plurality of rows of circumferentially extending rows of angled struts, with the rows being arrayed along the length of the frame between the outflow end 210 and the inflow end 212 of the frame 202. For example, the frame 202 can comprise a first row of angled struts 238 arranged end-to-end and extending circumferentially at the inflow end 212 of the frame; a second row of circumferentially extending, angled struts 236; a third row of circumferentially extending, angled struts 234; and a fourth row of circumferentially extending, angled struts 218 at the outflow end 210 of the frame 12. The fourth row of angled struts 218 can be connected to the third row of angled struts 234 by a plurality of axially extending window strut portions 240 and a plurality of axial (e.g., axially extending) struts 232. The axially extending window strut portions 240 define commissure windows (e.g., open windows) 242 that are spaced apart from one another around the frame 202, in a circumferential direction, and which are adapted to receive a pair of commissure tabs of a pair of adjacent leaflets 204 arranged into a commissure 230.

One or more (e.g., two, as shown in FIG. 3) axial struts 232 can be positioned between, in the circumferential direction, two commissure windows 242 formed by the window strut portions 240. Since the frame 202 can include fewer cells per row (e.g., nine) and fewer axial struts 232 between each commissure window 242, as compared to other prosthetic heart valves, such as the prosthetic heart valve 10 of FIG. 1, each cell 208 can have an increased width (in the circumferential direction), thereby providing a larger opening for blood flow and/or coronary access, as described herein.

Each axial strut 232 and each window strut portion 240 extends from a location defined by the convergence of the lower ends (e.g., ends arranged inward of and farthest away from the outflow end 210) of two angled struts 218 (which can also be referred to as an upper strut junction or upper elongated strut junction) to another location defined by the convergence of the upper ends (e.g., ends arranged closer to the outflow end 210) of two angled struts 234 (which can also be referred to as a lower strut junction or lower elongate strut junction). Each axial strut 232 and each window strut portion 240 forms an axial side of two adjacent cells of the first row of cells 214.

In some examples, as shown in FIG. 3, each axial strut 232 can have a width 244 that is larger than a width of the angled struts 218, 234, 236, and/or 238. As used herein, a “width” of a strut is measured between opposing locations on opposing surfaces of a strut that extend between the radially facing inner and outer surfaces of the strut (relative to the central longitudinal axis 216 of the frame 202). A “thickness” of a strut is measured between opposing locations on the radially facing inner and outer surfaces of a strut and is perpendicular to the width of the strut. In some examples, the width 244 of the axial struts 232 is 50-200%, 75-150%, or at least 100% larger than (e.g., double) the width of the angled struts of the frame 202. For example, if the angled struts 218, 234, 236, and 238 are approximately 0.3 mm wide, then the width 244 of the axial struts 232 can be in a range of 0.45 mm-0.9 mm, 0.5 mm-0.75 mm, or at least 0.6 mm.

For example, in known prosthetic heart valves (such as the valve 10 shown in FIG. 1) the axial struts have a same width as the other struts (e.g., angled struts) of the frame. As such, when the leaflets of the known prosthetic heart valve are pressed against the frame during the systolic phase, the leaflets may bend around the struts and/or extend radially outwardly through the cell openings due to the relatively narrow width of the axial struts. This phenomenon can reduce long-term durability of the leaflets, especially when the upper (e.g., outflow/free) edges of the leaflets are pushing against the axial struts and are bent over, as described above.

Thus, by providing the axial struts 232 with the width 244 that is greater than the width of other struts (e.g., angled struts of the frame 202), a larger contact area is provided for when the leaflets 204 contact the wider axial struts 232 during systole. This can, for example, distribute the stress and reduce the extent to which the leaflets 204 fold over the axial struts 232 and/or extend radially outward through the cells 208. As a result, one advantage of the disclosed technology is that the long-term durability of the leaflets 204 is increased.

In some cases, the free edges at the outflow end 228 of the leaflets 204 may press against the axial struts 232 at their outflow (e.g., upper) end portions 246. Accordingly, in some examples, the outflow end portions 246 of the axial struts 232 can be even wider than the width 244, which is depicted at an intermediate location of the axial strut 232 in FIG. 3, thereby providing an even larger area of contact and support for the leaflets 204.

As introduced above, since the frame 202 can have fewer cells in the circumferential direction (e.g., nine in the example depicted in FIG. 3) compared to the prosthetic valve of FIG. 1, each cell 208 can have an increased width (measured in the circumferential direction). This increased width of the cells 208 of the first row of cells 214 can enable the wider axial struts 232 to be incorporated into the frame 202, without sacrificing open space for blood flow and/or coronary access.

Commissure tabs of adjacent leaflets 204 can be secured together to form commissures 230. Each commissure 230 of the prosthetic heart valve 200 comprises two commissure tabs paired together, one from each of two adjacent leaflets 204, and extending through a commissure window 242 of the frame 202. Each commissure 230 can be secured to the window strut portions 240 forming the commissure window 242.

The cusp edge portion (e.g., scallop edge) of each leaflet 204 can be secured to the frame via one or more fasteners (e.g., sutures). In some examples, as shown in FIG. 3, the cusp edge portion of each leaflet 204 can be secured directly to the struts of the frame 202 (e.g., angled struts 234, 236, and 238). For example, the cusp edge portions of the leaflets 204 can be sutured to the angled struts 234, 236, 238 that generally follow the contour of the cusp edge portions of the leaflets.

In some examples, the cusp edge portion of the leaflets 204 can be secured to an inner skirt and the inner skirt can then be secured directly to the frame 202.

Further, in some examples, an outer skirt can be connected to an outer surface of the frame 202 (e.g., similar to the outer skirt 18 of the valve 10 of FIG. 1).

As shown in FIG. 3, in some examples, one or more of or each of the axial struts 232 can comprise an inflow end portion (e.g., inflow end portion that is closer to the inflow end than the outflow end portion 246) 248 that is widened relative to a middle portion 247 of the axial strut 232 (which can be defined by the width 244), similar to the outflow end portion 246 (as described above). In some examples, the inflow end portion 248 of the axial strut 232 can comprise an aperture 249. The apertures 249 can be configured to receive fasteners (e.g., sutures) for attaching soft components of the prosthetic heart valve 200 to the frame 202. For example, in some instances, an outer skirt can be positioned around an outer surface of the frame 202 and secured to the apertures 249 (e.g., as shown in FIG. 13 and described further below).

The frame 202 can further comprise a plurality of apices 220 formed at the inflow end 212 and the outflow end 210, each apex 220 forming a junction between two angled struts 218 at the inflow end 212 or outflow end 210. As such, the apices 220 are spaced apart from one another, in a circumferential direction at the inflow end 212 and the outflow end 210. As shown in FIG. 3, each apex 220 can have side portions 222 that curve or bend axially outward from the angled strut 218 to which it is connected and an end portion 224 that extends between the two side portions 222 of the apex 220. The side portions 222 can extend in a direction that is parallel to the central longitudinal axis 216. The end portion 224 can be relatively flat and include a surface that is disposed normal to the central longitudinal axis 216. Each apex 220 can have two bends at its end portion 224 and two bends at the side portions 222 (e.g., one at the junction between each side portion 222 and angled strut 218). In this way, the apices 220 can be U-shaped, similar to the apices 26 of the valve of FIG. 1.

While the apices 220 can be configured to distribute stresses at the apices 220 across the angled struts 218 to which they are connected, such a shape results in adding the height (in the axial direction) 221 of the apices 220 at the inflow end 212 and the outflow end 210 to the overall height of the prosthetic heart valve 200. As a result, the inflow end 226 of the leaflets 204 are spaced away from the inflow end 212 of the frame 202. This, in turn, can cause the outflow end 228 of the leaflets 204 to be disposed closer to the outflow end 210 of the frame, thereby leaving less open space in the cells 208 of the upper row of cells 214 between the outflow edges of the leaflets and the upper row of struts 218. In some examples, this arrangement can result in the leaflets 204, including commissures 230 of adjacent commissure tabs of the leaflets 204, to at least partially block blood flow into the coronary ostia (e.g., when the prosthetic heart valve is implanted in the native aortic valve). Further, coronary re-access devices can be inhibited from passing through such small spaces.

As introduced above, in examples where the prosthetic heart valve is implanted at the native aortic valve, in some instances an inflow end of the frame of the prosthetic heart valve can extend into the left ventricle outflow tract (LVOT) and can interact with the left ventricular wall at the LVOT level. Such interaction between the prosthetic heart valve and the native anatomy can, in some examples, cause conductance disturbances in the heart.

Thus, it can be advantageous to configure a prosthetic heart valve such that inflow ends of the leaflets (e.g., an inflow end of a cusp edge portion of the leaflet) extend below an inflow end or structural inflow end of a frame of the prosthetic heart valve, as described further below.

In some examples, arranging the leaflets of a prosthetic heart valve to be positioned below or distal to an inflow end of a frame of the prosthetic heart valve and further away from an outflow end of the frame can be achieved by configuring the frame to have inflow support struts that are non-structural to the frame and positioned below or distal to an inflow end of the frame, as defined by structural struts at the inflow end of the frame.

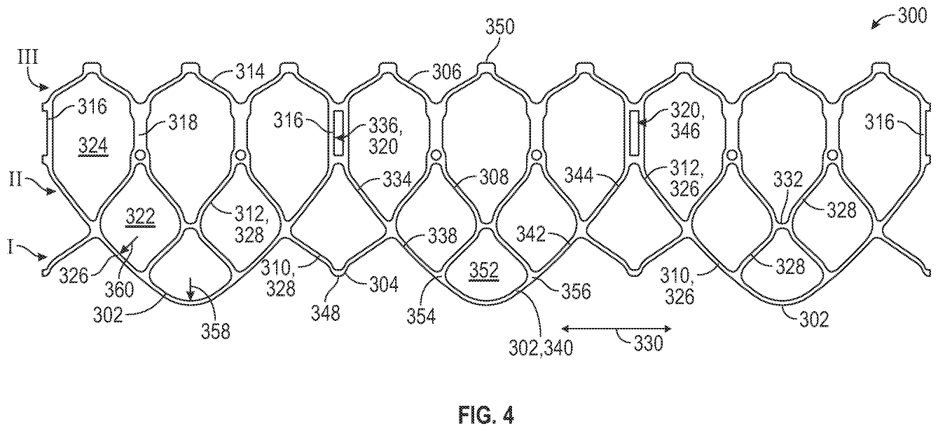

As used herein “non-structural to the frame,” in reference to struts of the frame, can refer to struts that are not required for normal radial expansion and compression of the frame. For example, if the frame did not include the “non-structural” inflow support struts, it could still radially expand and compress as intended (e.g., as required for deployment at an implantation site). Further, “structural” struts of the frame can refer to angled struts of the frame that are interconnected with one another and enable the frame to be radially compressed for delivery to an implantation site (on a delivery apparatus) and to be radially expanded and implanted at the implantation site. The structural struts can be configured to provide structure to the frame in a radially expanded configuration. In contrast, the non-structural struts may not provide structure or support that maintains the frame in the radially expanded configuration. Details on the structure of the inflow support struts are described further below with reference to FIGS. 4-6B

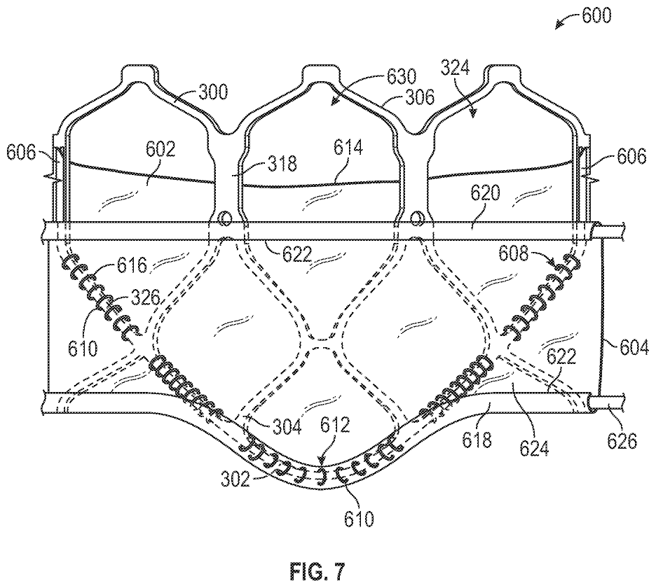

FIG. 4 shows an example of a frame 300 for a prosthetic heart valve, shown in a flattened configuration, which includes a plurality of inflow support struts 302 configured to support inflow ends of a plurality of leaflets (e.g., leaflets 204 shown in FIG. 3). The frame 300 has an inflow end 304 (a structural inflow end, as described further below) and an outflow end 306. A circumferential direction 330 is shown for reference in FIG. 4, indicating a circumferential direction of the frame 300 when the frame 300 is in its annular configuration (e.g., when left and right ends of the frame 300 are connected, similar to as shown in FIG. 3 and as shown in FIG. 7).

The frame 300 can comprise a plurality of interconnected struts 308 arranged in a plurality of rows of circumferentially extending (when the frame 300 is in an annular configuration, such as that shown in FIG. 3) rows of struts, with the rows being arrayed along the length of the frame between the outflow end 306 and the inflow end 304 of the frame 300. For example, the frame 300 can comprise a first row I of struts 310 arranged end-to-end and extending circumferentially at (and forming) the inflow end 304 of the frame 300; a second row II of struts 312 arranged end-to-end and extending circumferentially around the frame 300; and a third row III of struts 314 arranged end-to-end and extending circumferentially at (and forming) the outflow end 306 of the frame 300.

In some examples, the frame 300 can further comprise a plurality of inflow apices 348 that are formed at the inflow end 304 and spaced circumferentially apart from one another and a plurality of outflow apices 350 that are formed at the outflow end 306 and spaced circumferentially apart from one another. Each inflow apex 348 can be formed at a junction between two struts 310 of the first row I. Each outflow apex 350 can be formed at a junction between two angled struts 314 of the third row III. Thus, the inflow end 304 of the frame 300 can be defined and formed by the plurality of inflow apices 348 and the first row of struts 310 and the outflow end 306 of the frame 300 can be defined and formed by the plurality of outflow apices 350 and the third row III of struts 314.

The interconnected struts 308 form multiple rows of open cells between the outflow end 306 and the inflow end 304 of the frame 202. In some examples, as shown in FIG. 4, the frame 300 can comprise two rows of cells between the outflow end 306 and the inflow end 304. For example, the frame 300 can include a first row of cells 322 disposed at the inflow end 304 and a second row of cells 324 disposed at the outflow end 306. In some examples, the cells 322 of the second row can be elongated in an axial direction (relative to a central longitudinal axis of the frame 300), as compared to the cells 322 in the first row. For example, the cells 324 of the second row of cells can have a longer axial length, defined in a direction of a central longitudinal axis of the frame 300, than cells 322 in the first row.

The rows of cells of the frame 300 can comprise a plurality of cells (e.g., cells 322 or cells 324) arranged consecutively along and around the circumference of the frame 300.

In some examples, as shown in FIG. 3, each row of cells comprises nine cells.

In other examples, the frame 300 can comprise more than three rows of cells (e.g., four or five) and/or more or less than nine cells per row. Similarly, in some examples, the frame 300 can comprise more than three rows of struts (e.g., four or five). In some examples, the cells 324 in the second row of cells may not be elongated compared to cells 322 in the first row of cells.

The third row III of struts 314 can be connected to the second row II of struts 312 by a plurality of axially extending window strut portions 316 (similar to window strut portions 240 of FIG. 3) and a plurality of axial (e.g., axially extending) struts 318 (similar to the axial struts 232 of FIG. 3). The axially extending window strut portions 316 define commissure windows 320 (e.g., open windows) that are spaced apart from one another around the frame 300 and which are adapted to receive a pair of commissure tabs of a pair of adjacent leaflets arranged into a commissure (e.g., commissure tabs 250 of leaflets 204 in FIG. 3).

One or more (e.g., two, as shown in FIG. 4) axial struts 318 can be positioned between, in the circumferential direction 330, two commissure windows 320 formed by the window strut portions 316. Similar to the frame 202 of FIG. 2, in some examples, the frame 300 can include fewer cells per row (e.g., nine) and fewer axial struts 318 between each commissure window 320, as compared to other prosthetic heart valves, such as the prosthetic heart valve 10 of FIG. 1.

The interconnected struts 308 of the first row I of struts 310 and the second row II of struts 312 can include a plurality of cusp edge portion (or scallop) support struts 326 which extend from the axially extending window strut portions 316 to the inflow end 304 and the inflow support struts 302. The cusp edge portion support struts 326 can be configured to follow a cusp edge portion (or scallop line) of the leaflets secured to the frame. For example, as shown in FIG. 3 (and similarly in FIG. 7, as described further below) the cusp edge portion 252 of each leaflet 204 can extend, along a curved or U-shaped path, along the frame from one commissure window 242, to the inflow end 212, and to an adjacent commissure window 242.

Thus, in some examples, as shown in FIG. 4, the cusp edge portion support struts 326 can be configured (e.g., shaped) to follow a shape of the cusp edge portion of the leaflets and support the cusp edge portion of the leaflets. As described further below with reference to FIG. 7, in some examples, the leaflets can be secured (e.g., sutured) directly to the cusp edge portion support struts 326, instead of to an inner skirt, as can be done in prosthetic heart valves not including cusp edge portion support struts (e.g., the prosthetic heart valve 10 of FIG. 1). Thus, in some examples, as shown in FIG. 4, the cusp edge portion support struts 326 can be curved and follow a curved and/or undulating path between the commissure windows 320 and inflow support struts 302, around a circumference of the frame.

In contrast, the interconnected struts 308 of the first row I of struts 310 and the second row II of struts 312 can further include angled linear struts 328 that are relatively straight (e.g., straight between strut junctions 332 connecting adjacent struts and/or along a majority of the length of the angled linear strut 328). All the struts of the third row III of struts 314 can be angled linear struts that are relatively straight (not curved along their length).

Each axial strut 318 can extend from a location defined by the convergence of the lower ends (e.g., ends arranged inward of and farthest away from the outflow end 306) of two struts 314 of the third row III to another location defined by the convergence of upper ends (e.g., ends arranged closer to the outflow end 306) of two angled linear struts 328 of the second row II of struts 312. Each window strut portion 316 extends from a location defined by the convergence of the lower ends of two struts 314 of the third row III to another location defined by the convergence of the upper ends of two cusp edge portion support struts 326. Each axial strut 318 and each pair of window strut portions 316 forming one commissure window 320 form an axial side of two adjacent cells of the second row of cells 324.

As an example, a first cusp edge portion support strut 334 of the second row II of struts 312 can extend from window strut portions 316 forming a first commissure window 336 to a second cusp edge portion support strut 338 of the first row I of struts 310. The second cusp edge portion support strut 338 extends to the inflow end 304 and a first inflow support strut 340. The first inflow support strut 340 then curves and extends (in a direction back toward the outflow end 306) to a third cusp edge portion support strut 342 of the first row I of struts 310, which then extends to a fourth cusp edge portion support strut 344 of the second row II of struts 312. The fourth cusp edge portion support strut 344 then extends and connects to window strut portions 316 forming a second commissure window 346. In FIG. 4, the second commissure window 346 and the first commissure window 336 are separated from one another, in the circumferential direction 330, by two axial struts 318. However, in other examples, the second commissure window 346 and the first commissure window 336 can be separated from one another by more or less than two axial struts 318 (e.g., three). In this way, together the cusp edge portion support struts 326 and the inflow support struts 302 form a curved and undulating path around the frame 300 (in the circumferential direction 330) that is configured to follow a path and shape of the cusp edge portions of the leaflets secured to the frame. Thus, the cusp edge portion support struts 326 and the inflow support struts 302 can support the cusp edge portions of the leaflets secured thereto, as explained in further detail below.

In some examples, the inflow support struts 302 can also be referred to as cusp edge portion support struts (e.g., they may be a subset of the cusp edge portion support struts 326).

Each inflow support strut 302 can connect to and extend (and curve) between two adjacent cusp edge portion support struts 326 in the first row I of struts 310. For example, as shown in FIG. 4, the first inflow support strut 340 can couple to and extend between the second cusp edge portion support strut 338 and the third cusp edge portion support strut 342.

Further, each inflow support strut 302 can couple directly to and extend between two adjacent inflow apices (or junctions) 348. Such inflow apices 348 can form junctions between an inflow support strut 302, a cusp edge portion support strut 326 of the first row I of struts 310, and an angled linear strut 328 of the first row I of struts 310.

For example, as shown in FIG. 4, each inflow support strut 302 can curve from a first end 354 of the inflow support strut 302 to a second end 356 of the inflow support strut 302. The first end 354 can be connected to a first junction (inflow apex 348) between a first cusp edge portion support strut 326 and a first angled linear strut 328 of the first row I of struts 310. The second end 356 can be connected to a second junction (inflow apex 348) between a second cusp edge portion support strut 326 and a second angled linear strut 328 of the first row I of struts 310. In some examples, the inflow support strut 302 can continuously curve from its first end 354 to its second end 356 and have a radius of curvature 358.

As shown in FIG. 4, the inflow support struts 302 extend distally beyond the inflow end 304 of the frame 300. As used herein, the inflow end 304 of the frame 300 is defined by the inflow apices 348 and the structural struts of the frame 300 which are configured to allow the frame 300 to radially expand and compress into an expanded configuration and compressed configuration, respectively. For example, the inflow end 304 can be defined by the inflow apices 348, the angled linear struts 328 of the first row I of struts 310, and the cusp edge portion support struts 326 of the first row I of struts 310, which form a continuous line of struts (arranged end-to-end) in the circumferential direction and an undulating line in the axial direction.

In some examples, the inflow support struts 302 can be formed as integral struts of the frame 300 (e.g., formed or manufactured as one piece with a remainder of the struts of the frame). In other examples, the inflow support struts 302 can be rigidly affixed to the frame 300 (e.g., via welding, fasteners, and/or other means for rigidly affixing).

As shown in FIG. 4, the inflow support struts 302 are not connected together and do not form a continuous row of struts arranged end-to-end around the frame 300 (in the circumferential direction 330). Instead, the inflow support struts 302 are spaced apart from one another (discontinuous) around the frame 300 and form a third, non-continuous row of cells 352. Each cell 352 can be formed by one inflow support strut 302 and two adjacent angled linear struts 328 of the first row I of struts 310.

In some examples, as shown in FIG. 4, the frame 300 can include three inflow support struts 302, which results in three cells 352. The total number of inflow support struts 302 can equal a total number of leaflets of the prosthetic heart valve which is formed by the frame 300 (e.g., three leaflets). Thus, in some examples, the frame 300 can comprise exactly three inflow support struts 302. In other examples, if the prosthetic heart valve including the frame 300 includes more or less than three leaflets (e.g., two, four, or the like), the frame 300 can include more or less than three inflow support struts 302 (e.g., two, four, or the like, respectively).

The inflow support struts 302 are each configured to support an inflow end of a cusp edge portion (which in some examples can also be referred to as a scallop edge or edge portion) of a leaflet of the prosthetic heart valve. For example, as described above with reference to FIG. 3, each leaflet of a prosthetic heart valve (e.g., leaflet 204 shown in FIG. 3) can comprise a cusp edge portion (e.g., cusp edge portion 252 shown in FIG. 3) extending between opposing commissure tabs (e.g., commissure tabs 250 shown in FIG. 3) and the cusp edge portion can have an inflow end (or edge) (e.g., inflow end 226 shown in FIG. 3). The inflow end of the leaflet can be a central portion of the cusp edge portion. In some examples, the inflow end of each leaflet can be secured to and supported by a corresponding inflow support strut.

Thus, the inflow support struts 302 can be shaped such that they follow a curve of the inflow ends of the leaflets to be attached to the frame 300. As such, an amount of curvature or the radius of curvature 358 of the inflow support struts 302 can be smaller or larger than that shown in FIG. 4, such that they match or approximate the amount of curvature (e.g., radius of curvature) of the inflow ends of the leaflets. In some examples, the inflow support struts 302 can be referred to as having an arcuate shape.

Similarly, the shape and amount of curvature of the cusp edge portion support struts 326 can be configured to match or approximate a shape and amount of curvature of the corresponding portions of the cusp edge portions of the leaflets which attach thereto.

The cusp edge portion of the leaflets of the prosthetic heart valve including the frame 300 can be secured (e.g., sutured) directly to the cusp edge portion support struts 326 and inflow support struts 302 of the frame 300 (e.g., without an intervening inner skirt therebetween). Examples of such attachment are shown in FIGS. 7-10, as described below. Thus, the cusp edge portion support struts 326 and inflow support struts 302 are configured to support a load of the leaflets on the frame 300.

When the leaflets are secured to the frame 300, with the inflow ends of the leaflets attached to the inflow support struts 302, the inflow ends of the cusp edge portions of the leaflets extend distally beyond (e.g., past or outside of) the inflow end 304 of the frame. This advantageously results in the frame 300 being offset upward (i.e., proximally) relative to the distal-most portion of the leaflets (e.g., the inflow ends of the cusp edge portions), thereby reducing the frame's interaction with the left ventricular wall at the LVOT level when implanted in the native aortic valve annulus. This can, for example, reduce the likelihood of electrical conductance disturbances in the heart.

The curvature or shape of the cusp edge portion support struts 326 can be a gradual inclination that is configured to follow a line (e.g., scallop line) of the cusp edge portion of the leaflets. For example, in some instances, the curvature or shape of the cusp edge portion support struts 326 can deviate only a small amount from the straight or linear shape of the angled linear struts 328. As an example, the cusp edge portion support struts 326 can have a radius of curvature 360 that is smaller than the radius of curvature 358 of the inflow support struts 302 but greater than a straight or linear portion of the angled linear struts 328 (in some examples, by a relatively small amount such as by 5-15%). For example, the angled linear struts 328 can be straight along a majority of their length (e.g., a central portion of the angled linear strut that extends between the strut junctions 332). As such, the relatively small variance in shape from the angled linear struts 328 allows the cusp edge portion support struts 326 to contribute to the structure of the frame 300 without inhibiting the radial expansion and compression of the frame 300.

Since the shape of the inflow support struts 302 deviates to a greater degree from the traditional shape of the angled linear struts 328 (e.g., they have a greater radius of curvature 358 or amount of curvature and an apex or apex region), the inflow support struts 302 can, in some examples, resist or interfere with proper expansion and compression of the frame 300. Thus, it may be desirable to alter a geometry of the inflow support struts 302 such that they are non-structural to the frame and do not inhibit or interfere with the radial expansion and compression of the frame 300.

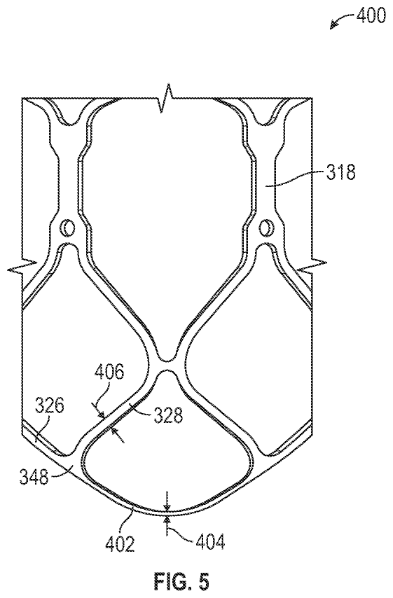

FIG. 5 illustrates one example of an inflow support strut 402 for a frame 400 which is configured with increased flexibility that reduces resistance to the radial expansion and compression of the frame 400. In some examples, the inflow support strut 402 is configured such that it does not interfere with the radial expansion and compression of the frame 400, while still being able to provide structural support to the inflow end of the leaflet secured thereto. The inflow support strut 402 can be further configured to reduce strains on the frame 400 during expansion and compression. In some examples, the inflow support strut 402 can be included as the inflow support struts of a frame similar to the frame 300 of FIG. 4.

As shown in FIG. 5, the inflow support strut 402, which has a curved or arcuate shape, can have a first width 404 that is smaller than a second width 406 of the structural struts of the frame (e.g., the cusp edge portion support struts 326 and/or the angled linear struts 328 to which it is coupled). In some examples, the inflow support strut 402 can have the narrower, first width 404 along its entire length (e.g., from a first end to an opposite, second end of the inflow support strut 402).

In some examples, both the cusp edge portion support struts 326 and the angled linear struts 328 of the frame 400 can have the same, second width 406.

In some examples, a width of the cusp edge portion support struts 326 can be smaller or larger than the second width 406 and the angled linear struts 328 can have the second width 406.

In some examples, the second width 406 can be in a range of about 0.35 to 0.45 mm and the first width 404 of the inflow support strut 402 can be in a range of about 0.1 to 0.25 mm. In some examples, the second width 406 can be in a range of about 0.25 to 0.4 mm and the first width 404 of the inflow support strut 402 can be in a range of about 0.1 to 0.25 mm or 0.1 to 0.2 mm.

The first width 404 can be selected such that the inflow support strut 402 has increased flexibility and is configured to bend at least at a central region of the inflow support strut 402.

By utilizing thinned inflow support struts 402 having the smaller first width 404 (as compared to the second width 406), the inflow support struts 402 can adequately support the inflow ends of the leaflets secured thereto without resisting (or offering little resistance to the) transformation of the frame 400 between a radially expanded and radially compressed configuration. The smaller first width 404 of the inflow support struts 402 can result in relatively flexible inflow support struts that are configured to bend during radial compression of the frame. The inflow support struts 402 are configured such that they do not inhibit the radial compression and expansion of the frame 400 and such that they reduce strains experienced by the frame 400 during compression and expansion due to the presence of the narrower inflow support struts 402 (as compared to the inflow support struts 302).

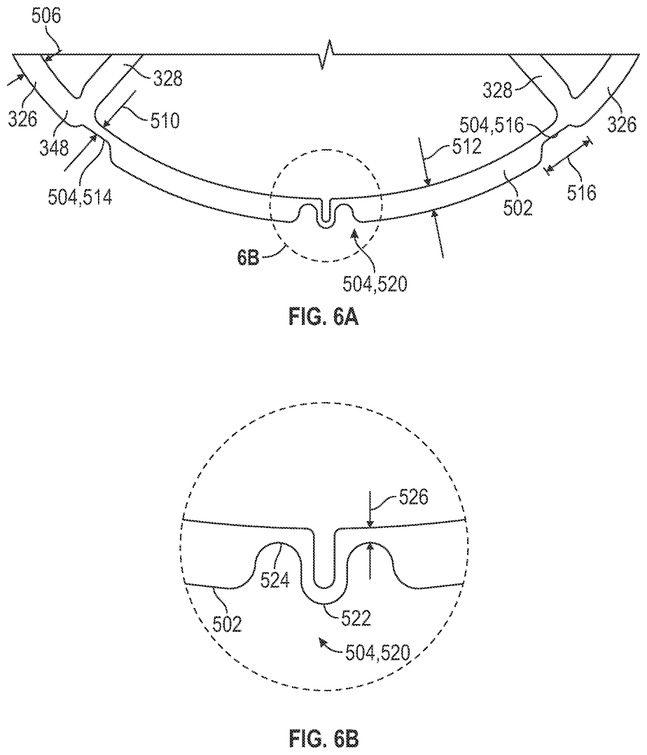

FIG. 6A-6B illustrate another example of an inflow support strut 502 for a frame of a prosthetic heart valve which is configured with increased flexibility that reduces resistance to the radial expansion and compression of the frame. In some examples, the inflow support strut 502 is configured such that it does not interfere with the radial expansion and compression of the frame, while still being able to provide structural support to the inflow end of the leaflet secured thereto. The inflow support strut 502 can be further configured to reduce strains on the frame during expansion and compression.

FIG. 6A shows one inflow support strut 502 of a frame for a prosthetic heart valve. In some examples, the inflow support strut 502 can replace each of the inflow support struts 302 of the frame 300 of FIG. 4. In other examples, the inflow support strut 502 can be used in another frame for a prosthetic heart valve. Each inflow support strut 502 can be configured to support an inflow end of a cusp edge portion of a leaflet.

In some examples, as shown in FIG. 6A, the inflow support strut 502 can be connected to cusp edge portion support struts 326 and angled linear struts 328 of the frame. For example, the inflow support strut 502 can be connected to two adjacent junctions (e.g., inflow apices 348) of the structural struts of the frame at the inflow end of the frame (e.g., the cusp edge portion support strut 326 and the angled linear strut 328).

As shown in FIG. 6A, the inflow support strut 502, which has a curved or arcuate shape, can have one or more thinned portions or regions 504. The thinned regions 504 can have a second width 510 (or thickness) that is smaller than a first width 506 of the structural struts of the frame (e.g., the cusp edge portion support struts 326 and/or the angled linear struts 328 to which the inflow support strut 502 is coupled). A remainder of the inflow support strut 502 (e.g., non-thinned portion or region) can have a third width 512 that is larger than the second width 510. In some examples, the third width 512 is the same as the first width 506. In other examples, the third width 512 is smaller than the first width 506.

In some examples, the second width 510 can be in a range of about 0.05 to 0.3 mm and the third width 512 can be in a range of about 0.5 to 1.0 mm. In some examples, the first width 506 of the structural (“traditional”) struts of the frame can be in a range of about 0.25 to 0.4 mm or 0.35 to 0.45 mm.

The one or more thinned regions 504 can include a first thinned region 514 and a second thinned region 516 formed at opposite ends of the inflow support strut 502 and connecting to a corresponding junction or inflow apex 348 of the frame. In this way, the inflow support strut 502 has the narrower second width 510 at its ends which connect or attach to the structural struts (e.g., the cusp edge portion support strut 326 and the angled linear strut 328) at the inflow end of the frame.

In some examples, a length (e.g., arc length) 518 of the first thinned region 514 and/or the second thinned region 516 can be in a range of about 0.2 to 0.4 mm, at least 0.2 mm, or at least 10% a total length of the inflow support strut.

The one or more thinned regions 504 can further (or alternatively) include a third thinned region 520. In some examples, the third thinned region 520 can be formed at a central (or middle) region of the inflow support strut 502. For example, the third thinned region 520 can be formed between the first thinned region 514 and the second thinned region 516. Thus, the third thinned region 520 can be referred to as a central thinned region of the inflow support strut 502. A detail view of the third thinned region 520 is shown in FIG. 6B.

In some examples the third thinned region 520 can have an undulating shape that forms one or more peaks 522 and valleys 524 in the inflow support strut 502 (e.g., two valleys 524 and one peak 522 in the example of FIGS. 6A and 6B). As a result, the inflow support strut 502 can be configured to bend at the third thinned region 520 during radial compression of the frame.

In some examples, the third thinned region 520 can have a fourth width 526, which can be the same as or different than the second width 510. However, even if the fourth width 526 is different than the second width 510, the fourth width 526 can be smaller than the third width 512 and the first width 506.

The thinned regions 504 are configured to reduce the residual strains on the inflow support strut 502 at the locations of attachment to the junctions (e.g., inflow apices 348) and the bending at the central region of the inflow support strut 502, during radial compression and expansion of the frame.

A thickness of the inflow support strut 502 in the radial direction can be the same as the structural struts of the frame. As a result, the inflow support strut 502 can carry the radial loads exerted thereon by the cusp edge portion of the leaflets attached thereto during diastole. This can result in the inflow support strut 502 retaining its load-bearing capacity in the radial direction, while being thinned only along a plane that does not carry significant loads during operation of the prosthetic heart valve in vivo.

The configurations of the frames described above with reference to FIGS. 4-6B enable leaflets of a prosthetic heart valve to be secured directly to the cusp edge portion support struts and the inflow support struts of the frame of the prosthetic heart valve. For example, instead of securing the leaflets to an inner skirt which is secured to an inner surface of the frame (e.g., as shown in FIG. 1), the prosthetic heart valve may not include the inner skirt and the leaflets can be attached (e.g., sutured) directly to the frame. As described above, the struts to which the leaflets are directly sutured (the inflow support struts and the cusp edge portion support struts) are configured to support a load of the leaflets while not interfering with radial compression and expansion of the frame. For example, the inflow support struts can have increased flexibility relative to the remaining, structural struts of the frame, thereby reducing their resistance to radial expansion and compression of the frame. Examples of prosthetic heart valves comprising leaflets secured directly to the frame, including inflow support struts, and only an outer skirt (e.g., no inner skirt disposed between the frame and the leaflets) are shown in FIGS. 7-10, which are described in detail below.

FIG. 7 shows a side view of a portion of a prosthetic heart valve 600 comprising the frame 300 with inflow support struts 302. For example, FIG. 7 shows three cells at the outflow end 306 of the frame 300 and one inflow support strut 302 extending between cusp edge portion support struts 326 which extend between two adjacent commissure windows 320 of the frame (only a front portion of the frame 300 is shown and a rear portion of the frame is removed for the sake of illustration). In some examples, the inflow support struts 302 can be configured to be the same as the inflow support strut 402 of FIG. 5 or the inflow support strut 502 of FIGS. 6A-6B. FIG. 7 shows one leaflet 602 attached to the frame 300 and an outer skirt 604 disposed around an outer surface of the frame 300.

Specifically, as shown in FIG. 7, opposing commissure tabs 606 of the leaflet 602 are secured to adjacent commissure windows 320 of the frame 300. Further, a cusp edge portion 608 of the leaflet 602 is secured directly to the cusp edge portion support struts 326 and the inflow support strut 302 via a plurality of sutures 610 (or other attachment members). For example, an inflow end 612 of the leaflet 602 is secured directly to the inflow support strut 302 via sutures 610. As a result, the inflow end 612 of the leaflet 602 is offset from and disposed below (or distal to) the inflow end 304 of the frame 300 (FIG. 7). Additionally, an outflow edge 614 of the leaflet 602 is offset from and disposed distal to the outflow end 306 (and toward the inflow end 304) of the frame 300, thereby creating open space 630 in the cells 324, between the outflow edge 614 and the outflow end 306 of the frame. As a result, serviceability of and/or coronary access through the open spaces 630 of the prosthetic heart valve 600 is increased (as compared to the prosthetic valve 10 which does not have leaflets that are offset from the outflow end of the frame).

The outer skirt 604 is disposed over and around an outer surface of the frame 300 and can be secured to the struts of the frame 300 with one or more attachment members (e.g., sutures). In contrast, the leaflets 602 are arranged on an inner surface of the frame 300. The outer skirt 604 is depicted in FIG. 7 as semi-transparent, showing the underlying components of the frame 300 in dashed lines for illustration purposes. For example, struts of the frame 300 that are disposed between an inflow end 618 (or edge) and an opposite, outflow end 620 of the outer skirt 604 are depicted with dashed lines to denote their positioning underneath (interior to) the outer skirt 604.

In some examples, the outer skirt 604 can be secured to the frame 300 with the sutures 610 which also secure the leaflets 602 to the frame 300. As such, the sutures 610 can connect both the leaflets 602 and the outer skirt 604 to the cusp edge portion support struts 326 and the inflow support strut 302.

In some examples, the individual stitches of the sutures 610 can be floating or loose stitches 616 (or sutures) that are configured to be relatively loose and allow the outer skirt 604 to float or slide along the struts it is sutured to without being stretched in a longitudinal direction when in the compressed configuration. Additional details on such loose stitches can be found in U.S. Pat. No. 10,610,362, which is incorporated by reference herein.

In other examples, the sutures 610 can secure the leaflets 602 to the frame 300, as described above, and the outer skirt 604 can be secured to the outer surface of the fame with additional sutures (or other attachment means), which in some examples can comprise one or more loose stitches or other types of stitches (such as those shown securing the outer skirt 18 to the frame in FIG. 1).

In some examples, since the leaflets 602 are directly attached to the cusp edge portion support struts 326 and inflow support strut 302, the outer skirt 604 can comprise a more thromboresistant material, such as ePTFE instead of PET (which inner skirts, such as the inner skirt 16 of FIG. 1, can be commonly comprised of). Thus, the outer skirt 604 can be configured as a perivalvular outer sealing member that is configured to reduce perivalvular leakage when the prosthetic heart valve 600 is implanted at a native heart valve.

For example, inner skirts for prosthetic heart valves can traditionally comprise PET which has increased strength for supporting the leaflets secured thereto. In some cases, ePTFE lacks the material strength offered by PET. However, since a skirt of the prosthetic heart valve 600 is not required to carry loads of the leaflets 602, utilization of ePTFE for the outer skirt 604 (which can be an only skirt of the prosthetic heart valve 600, as shown in FIG. 7) is possible. Since ePTFE is less prone to tissue ingrowth than PET, this can result in improved long-term durability of the prosthetic heart valve 600. For example, when the prosthetic heart valve is implanted, interactions between the outer skirt and the surrounding tissue may trigger an adverse tissue response that can result in thrombus formation in the short term and/or pannus formation in the long term. However, by having an outer skirt that comprises a more thromboresistant material like ePTFE, such adverse tissue responses can be reduced.

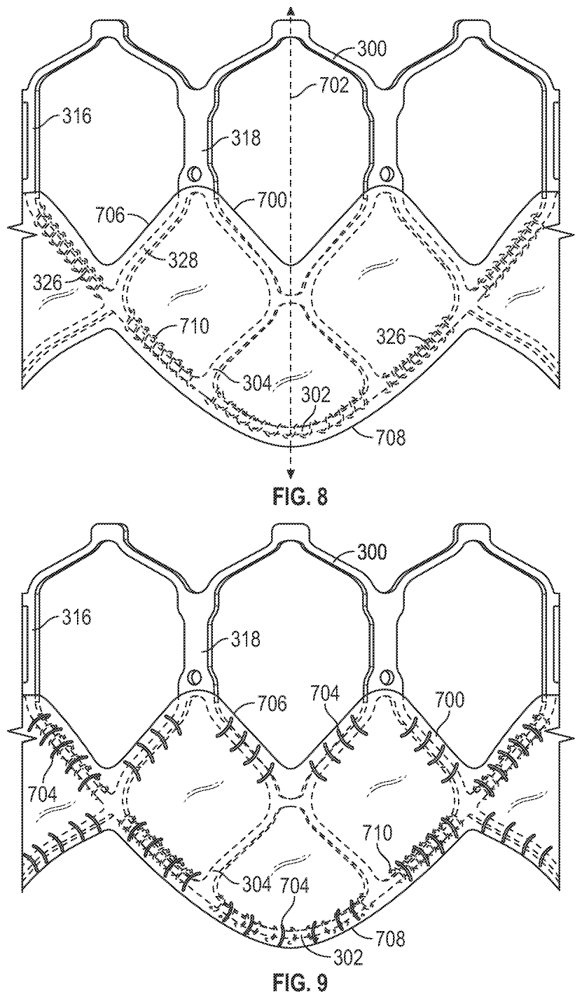

FIGS. 8 and 9 depict another example of an outer skirt 700 for a prosthetic heart valve which is configured to be arranged around an outer surface of the frame 300 (or another frame of a prosthetic heart valve). FIGS. 8 and 9 show a portion of the frame 300 with the outer skirt 700 disposed around the outer surface of the frame 300. Components of the frame 300 that are covered by the outer skirt 700 are depicted with dashed lines in FIGS. 8 and 9 for the purposes of illustration. As explained further below, FIG. 9 depicts attachment members (e.g., sutures) used to attach the outer skirt 700 to the frame 300.

The outer skirt 700 can be configured such that it does not elongate when the frame 300 is radially compressed. For example, in some instances, the yarns (e.g., warp and weft yarns) of the fabric of the outer skirt 700 can be arranged perpendicular to one another and at 0° and 90°, respectively, from a longitudinal axis 702 of the prosthetic heart valve. Such an arrangement of the fabric of the outer skirt 700 can make it resistant to elongation (longitudinally) when the frame 300 is radially compressed. Instead of changing the arrangement of fibers of the outer skirt 700, the outer skirt 700 can be attached to the frame 300 such that the frame 300 can be radially compressed without stretching and elongating the outer skirt 700 in the longitudinal direction.

For example, as shown in FIG. 9, the outer skirt 700 can be attached to the frame 300 by a plurality of floating or loose stitches 704 (or sutures), thereby allowing the outer skirt 700 to “float” along the struts of the frame 300 without being stretched in the longitudinal direction in the compressed configuration, as described above with reference to the loose stitches 616 of FIG. 7.

For example, as shown in FIG. 9, the outer skirt 700 can be attached by the floating or loose stitches 704 to the struts of the frame 300. The loose stitches 704 can secure the outer skirt 700 to the cusp edge portion support struts 326, the angled linear struts 328, and/or the inflow support struts 302 that are disposed adjacent to and underneath an outflow end 706 (or edge) and an inflow end 708 (or edge) of the outer skirt 700. Thus, in some examples, as shown in FIG. 9, the inflow end 708 and the outflow end 706 of the outer skirt 700 can be secured to the struts of the frame 300, on an outside of the frame 300, by the loose stitches 704. In some examples, a central portion of the outer skirt 700 can be free and unattached to the struts of the frame 300.

In some examples, as shown in FIGS. 8 and 9, the loose stitches 704 can be separate from sutures 710 (or stitches) that secure the leaflets to the frame. In some examples, the sutures 710 can be configured similarly to those of sutures 610 in FIG. 7 but may not connect to the outer skirt 700. The leaflets are omitted from FIGS. 8 and 9 for the ease of illustration (however they may be arranged similarly on the frame 300 as shown in FIG. 7).

Similar to the outer skirt 604 of FIG. 7, in some examples, the outer skirt 700 can comprise ePTFE.

In some examples, as shown in FIGS. 8 and 9, the outflow end 706 and/or the inflow end 708 of the outer skirt 700 can have an undulating shape. For example, in some instances, the outflow end 706 can undulate and follow the angling (e.g., zig-zag) of the cusp edge portion support struts 326 and angled linear struts 328 that extend from the axial struts 318 and/or the window strut portions 316 and that are disposed in a same circumferentially extending row of the frame 300 (e.g., the struts 312 of the second row II of struts in FIG. 4). Additionally or alternatively, in some examples, the inflow end 708 can undulate and follow the angling of the inflow support struts 302, the cusp edge portion support struts 326, and the angled linear struts 328 that form the inflow end 304 of the frame 300 (e.g., the first row I of struts 310 shown in FIG. 1 and the inflow support struts 302). Due to its coupling to the inflow support struts 302, the outer skirt 700 can also extend beyond the inflow end 304 of the frame 300.

Returning to FIG. 7, according to some examples, in order to avoid direct contact between the leaflets and edges 622 of the outer skirt 604 (which can be relatively sharp and/or abrasive), the inflow end 618 and/or the outflow end 620 of the outer skirt 604 can be rolled over monofilaments 626, thereby forming a folded over edge at the inflow end 618 and/or outflow end 620 of the outer skirt 604. As a result, the edges 622 of the outer skirt 604 are disposed against an outer surface 624 of the outer skirt 604 and are prevented from contacting the leaflets 602 (which face an inner surface of the outer skirt 604). The resulting folded over edges of the inflow end 618 and/or the outflow end 620 can be more atraumatic and thus a likelihood of abrasion of the leaflets is reduced.

In some examples, the monofilaments 626 are radiopaque (for example, made of radiopaque materials, or coated by a radiopaque coating), so as to allow visibility thereof during the implantation procedure for proper alignment of the prosthetic heart valve 600 relative to an outflow portion of the native leaflets of the native heart valve.

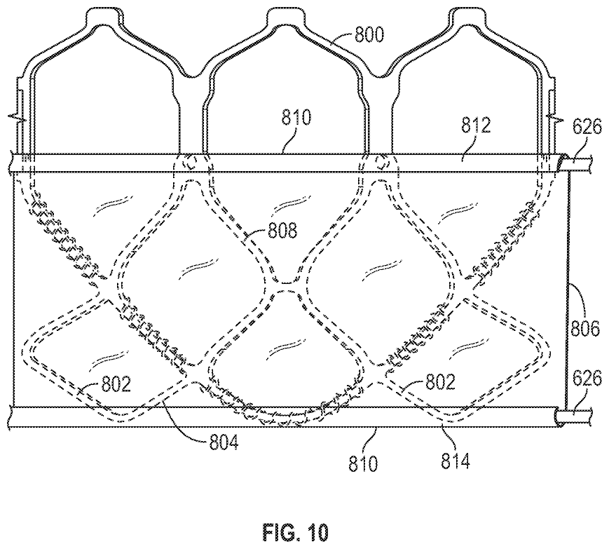

FIG. 10 shows another example of a frame 800 which can be similar to the frame 300, but it includes additional angled struts 802 that form an inflow end 804 of the frame 800. In some examples, the angled struts 802 form the first row of struts at the inflow end 804, along with the inflow support struts 302. However, in other examples, the frame 800 may not include inflow support struts.

An outer skirt 806 is disposed on and around an outer surface 808 of the frame 800. The outer skirt 806 can include folded over edges 810 which form an outflow end 812 and an inflow end 814 of the outer skirt 806. In this way, the outer skirt 806 can be similar to the outer skirt 604 of FIG. 7, except its inflow end 814 is relatively straight (due to the frame 800 including the additional angled struts 802). In contrast, the inflow end 618 of the outer skirt 604 of FIG. 7 is undulating to follow the inflow support struts 302 which extend distal to the inflow end 304 of the frame 300.

In this way, a frame having an outer skirt comprising a more thromboresistant material can result in less tissue ingrowth into and increased durability of the prosthetic heart valve. Further, by having a frame with inflow support struts that extend beyond the inflow end of the frame and that are non-structural to the frame (e.g., do not interfere with radial expansion and contraction of the frame), both the inflow and outflow edges of the leaflets can be offset distally with respect to the inflow and outflow ends of the frame. As a result, a serviceability (e.g., ability for future coronary access or unhindered valve-in-valve procedures) of the prosthetic heart valve can be improved and conductance disturbances in the heart can be reduced or avoided.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable and compressible annular frame comprising: a plurality of interconnected struts defining a plurality of rows of cells arranged between a first end and a second end of the frame, the plurality of interconnected struts comprising a plurality of first struts defining the first end of the frame; and a plurality of second struts axially offset from the first end of the frame, each second strut connected to and between two adjacent first struts of the plurality of first struts and extending axially away from the two adjacent first struts; and a plurality of leaflets attached to the frame, wherein each leaflet of the plurality of leaflets comprises a cusp edge portion and a free edge portion, and wherein an inflow end of the cusp edge portion of each leaflet of the plurality of leaflets is secured to a corresponding second strut of the plurality of second struts such that the inflow end is axially offset from the first end of the frame.

Example 2. The prosthetic heart valve of any example herein, particularly example 1, wherein the plurality of interconnected struts is configured as structural struts of the frame that support a radial expansion and compression of the frame and provide structure to the frame in a radially expanded configuration, and wherein the plurality of second struts is configured to be non-structural to the frame and provide support to inflow ends of the plurality of leaflets.

Example 3. The prosthetic heart valve of any example herein, particularly example 1 or example 2, wherein a total number of second struts of the plurality of second struts equals a total number of leaflets of the plurality of leaflets.

Example 4. The prosthetic heart valve of any example herein, particularly any one of examples 1-3, wherein the plurality of second structs is spaced apart from one another around a circumference of the frame.

Example 5. The prosthetic heart valve of any example herein, particularly any one of examples 1-4, wherein each second strut of the plurality of second struts curves from a first end of the second strut to a second end of the second strut, the first end connected to a first junction between a first pair of first struts of the plurality of first struts and the second end connected to a second junction between a second pair of first struts of the plurality of first struts.

Example 6. The prosthetic heart valve of any example herein, particularly example 5, wherein each second strut has a radius of curvature that follows a radius of curvature of the inflow end of the cusp edge portion of the leaflet.

Example 7. The prosthetic heart valve of any example herein, particularly example 5 or example 6, wherein each second strut curves by a greater amount than each first strut of the plurality of first struts.

Example 8. The prosthetic heart valve of any example herein, particularly any one of examples 1-7, wherein the plurality of second struts is configured to have increased flexibility relative to the plurality of interconnected struts to reduce resistance to radial expansion and compression of the frame.

Example 9. The prosthetic heart valve of any example herein, particularly example 8, where the plurality of second struts has a second width that is smaller than a first width of the plurality of first struts.

Example 10. The prosthetic heart valve of any example herein, particularly example 9, wherein the second width can be in a range of 0.1-0.25 mm and the first width can be in a range of 0.25-0.4 mm.

Example 11. The prosthetic heart valve of any example herein, particularly example 8, wherein each second strut of the plurality of second struts has one or more thinned regions which has a width that is smaller than a width of a remainder of the second strut and a width of the plurality of first struts.

Example 12. The prosthetic heart valve of any example herein, particularly example 11, wherein the one or more thinned regions includes end regions extending from a first end and a second end of the second strut toward a central region of the second strut.

Example 13. The prosthetic heart valve of any example herein, particularly example 11 or example 12, wherein the one or more thinned regions includes a central region of the second strut which has an undulating shape that forms one or more peaks and valleys in the second strut.

Example 14. The prosthetic heart valve of any example herein, particularly any one of examples 1-13, wherein, for each leaflet, the inflow end of the cusp edge portion of the leaflet is directly attached to the corresponding second strut such that the inflow end of the cusp edge portion of the leaflet is offset from the first end of the frame and an outflow edge of the leaflet is offset from the second end of the frame, toward the first end of the frame, and wherein the first end of the frame is an inflow end of the frame and the second end of the frame is an outflow end of the frame

Example 15. The prosthetic heart valve of any example herein, particularly any one of examples 1-14, wherein, for each leaflet, the inflow end of the cusp edge portion of the leaflet is secured directly to the corresponding second strut by one or more sutures.

Example 16. The prosthetic heart valve of any example herein, particularly example 15, further comprising an outer skirt disposed around an outer surface of the frame and wherein the one or more sutures attach both the inflow ends of the cusp edge portions of the leaflets and an inflow end of the outer skirt to the frame, the plurality of leaflets arranged on an inside of the frame.

Example 17. The prosthetic heart valve of any example herein, particularly any one of examples 1-16, wherein the plurality of first struts include a plurality of cusp edge portion support struts that extend from a corresponding second strut toward a commis sure window of the frame.

Example 18. The prosthetic heart valve of any example herein, particularly example 17, wherein the plurality of first struts include a plurality of angled linear struts, wherein the plurality of cusp edge portion support struts has a greater curvature than the plurality of angled linear struts, and wherein the greater curvature of the plurality of cusp edge portion support struts is configured to follow a curvature of the cusp edge portion of the plurality of leaflets.

Example 19. The prosthetic heart valve of any example herein, particularly any one of examples 1-18, wherein each leaflet comprises two commissure tabs disposed on opposite sides of the leaflet with the cusp edge portion extending between the two commissure tabs.

Example 20. The prosthetic heart valve of any example herein, particularly example 19, wherein the plurality of interconnected struts further comprises a plurality of commissure windows formed by struts of the plurality of interconnected struts that form cells of a first row of cells of the plurality of rows of cells, the first row of cells disposed at the second end of the frame, and wherein each commissure window is configured to receive commissure tabs of two adjacent leaflets of the plurality of leaflets.

Example 21. The prosthetic heart valve of any example herein, particularly example 20, wherein the cusp edge portion of each leaflet extends along and is secured directly to a first portion of the plurality of first struts that extend from a first commissure window to a corresponding second strut and to an adjacent, second commissure window.

Example 22. The prosthetic heart valve of any example herein, particularly example 21, wherein the first portion of the plurality of first struts are curved and configured to follow a curvature of the cusp edge portion of the leaflet.

Example 23. The prosthetic heart valve of any example herein, particularly example 21 or example 22, further comprising an outer skirt disposed around an outer surface of the frame.

Example 24. The prosthetic heart valve of any example herein, particularly example 23, wherein the outer skirt is attached to the frame by a plurality of loose stitches configured to allow the outer skirt to float along the first portion of the plurality of first struts as the prosthetic heart valve is radially compressed.

Example 25. The prosthetic heart valve of any example herein, particularly example 23 or example 24, wherein the outer skirt comprises ePTFE.

Example 26. The prosthetic heart valve of any example herein, particularly any one of examples 23-25, wherein the outer skirt has a first end that undulates between the first end of the frame and the plurality of second struts, around a circumference of the frame.

Example 27. The prosthetic heart valve of any example herein, particularly any one of examples 23-26, wherein the outer skirt has a first end and a second end arranged opposite one another, the first end disposed at the first end of the frame and wherein the first end and the second end of the outer skirt are rolled over monofilaments to form folded over edges at the first end and the second end of the outer skirt.

Example 28. The prosthetic heart valve of any example herein, particularly example 27, wherein the monofilaments are radiopaque.

Example 29. A prosthetic heart valve comprising: a radially expandable and compressible annular frame comprising: a plurality of interconnected struts defining a plurality of rows of cells arranged between an inflow end and an outflow end of the frame, the plurality of interconnected struts comprising: a plurality of first struts defining a plurality of commissure windows disposed adjacent to the outflow end of the frame; and a plurality of second struts arranged end-to-end and extending between the plurality of commissure windows and the inflow end of the frame in an undulating pattern around a circumference of the frame; and a plurality of third struts axially offset from the inflow end of the frame, each third strut of the plurality of third struts connected to two adjacent second struts of the plurality of second struts which form a part of the inflow end of the frame.

Example 30. The prosthetic heart valve of any example herein, particularly example 29, further comprising a plurality of leaflets attached directly to the frame, wherein each leaflet comprises two commissure tabs disposed on opposite sides of the leaflet and a cusp edge portion extending between the two commissure tabs, and wherein the cusp edge portion of each leaflet is secured directly to the plurality of second struts and the plurality of third struts.

Example 31. The prosthetic heart valve of any example herein, particularly example 30, wherein an inflow end of the cusp edge portion of each leaflet is secured directly to a corresponding third strut of the plurality of third struts and wherein each third strut of the plurality of third struts is curved between its ends such that each third strut follows a curvature of the inflow end of the cusp edge portion of the leaflet secured thereto.

Example 32. The prosthetic heart valve of any example herein, particularly example 31, wherein each second strut of the plurality of second struts is curved to follow a curvature of a portion the cusp edge portion of the leaflet secured directly thereto.

Example 33. The prosthetic heart valve of any example herein, particularly example 32, wherein a first radius of curvature of the plurality of third struts is greater than a second radius of curvature of the plurality of second struts.

Example 34. The prosthetic heart valve of any example herein, particularly any one of examples 30-33, further comprising an outer skirt disposed around an outer surface of the frame, the outer skirt having an inflow edge and outflow edge that are folded over and disposed against an outer surface of the outer skirt.

Example 35. The prosthetic heart valve of any example herein, particularly example 34, wherein the plurality of leaflets and the outer skirt are secured to the plurality of second struts and the plurality of third struts by a plurality of loose stitches.

Example 36. The prosthetic heart valve of any example herein, particularly any one of examples 29-35, wherein the two adjacent second struts extend from different commissure windows of two adjacent commissure windows of the plurality of commissure windows.

Example 37. The prosthetic heart valve of any example herein, particularly any one of examples 29-36, wherein the plurality of interconnected struts further comprises a plurality of fourth struts that are angled and straight along a majority of their length.

Example 38. The prosthetic heart valve of any example herein, particularly example 37, wherein each third strut is further connected to two adjacent fourth struts forming a part of the inflow end of the frame.

Example 39. The prosthetic heart valve of any example herein, particularly any one of examples 29-38, wherein at least a portion of each third strut has a first width that is smaller than a second width the plurality of second struts.

Example 40. The prosthetic heart valve of any example herein, particularly example 39, wherein an entirety of each third strut has the first width that is smaller than the second width of the plurality of second struts.

Example 41. The prosthetic heart valve of any example herein, particularly example 39, wherein opposite end portions of each third strut that are connected to the two adjacent second struts and a central portion of each third strut have the first width and wherein a remainder of each third strut has a third width that is greater than the first width.

Example 42. The prosthetic heart valve of any example herein, particularly example 41, wherein the central portion has an undulating shape that forms one or more peaks and one or more valleys along its length.

Example 43. A prosthetic heart valve comprising: a radially expandable and compressible annular frame comprising: a plurality of interconnected struts defining a plurality of rows of cells arranged between an inflow end and an outflow end of the frame and configured to support a radial expansion and compression of the frame, the plurality of interconnected struts including a plurality of first struts arranged end-to-end around a circumference of the frame and forming the inflow end of the frame; a plurality of apices formed at the inflow end of the frame, each apex of the plurality of apices formed at a junction between two adjacent first struts of the plurality of first struts; and a plurality of arcuate second struts that extend axially beyond the inflow end of the frame, where each second strut of the plurality of second struts is connected to and between two adjacent apices of the plurality of apices and is configured to bend at a central region of the second strut during radial compression of the frame.

Example 44. The prosthetic heart valve of any example herein, particularly example 43, wherein each second strut of the plurality of second struts comprises one or more regions that have a first width that is smaller than a second width of the plurality of first struts.

Example 45. The prosthetic heart valve of any example herein, particularly example 44, wherein each second strut has the smaller first width along an entire length of the second strut, from a first apex to a second apex of the plurality of apices to which the second strut is connected.

Example 46. The prosthetic heart valve of any example herein, particularly example 44, wherein each second strut has one or more thinned regions along a length of the second strut that has the first width and wherein the first width is smaller than a third width of a remainder of the second strut.

Example 47. The prosthetic heart valve of any example herein, particularly example 46, wherein the one or more thinned regions includes the central region of the second strut and wherein the central region undulates and forms one or more peaks and one or more valleys along the central region.

Example 48. The prosthetic heart valve of any example herein, particularly example 46 or example 47, wherein the one or more thinned regions includes end regions of the second strut that connect to the two adjacent apices.

Example 49. The prosthetic heart valve of any example herein, particularly any one of examples 42-48, further comprising a plurality of leaflets, each leaflet comprising a cusp edge portion extending between two commissure tabs of the leaflet, wherein an inflow end of the cusp edge portion of each leaflet is secured directly to a corresponding second strut of the plurality of second struts.

Example 50. The prosthetic heart valve of any example herein, particularly example 49, wherein a remainder of the cusp edge portion of each leaflet is secured directly to a first portion of the plurality of first struts that extend from commissure windows of the frame to the plurality of second struts, wherein the commissure windows are configured to receive commissure tabs of adjacent leaflets of the plurality of leaflets.

Example 51. The prosthetic heart valve of any example herein, particularly example 50, wherein each first strut of the first portion of the plurality of first struts is curved and configured to follow a shape of a portion of the cusp edge portion of the leaflet secured thereto.

Example 52. The prosthetic heart valve of any example herein, particularly any one of examples 49-51, wherein the plurality of leaflets comprises exactly three leaflets and the plurality of second struts comprises exactly three second struts.

Example 53. The prosthetic heart valve of any example herein, particularly any one of examples 49-52, further comprising an outer skirt disposed around an outer surface of the frame and having an inflow end that extends along the plurality of first struts and the plurality of second struts in an undulating pattern around the inflow end of the frame.

Example 54. A prosthetic heart valve comprising: a radially expandable and compressible annular frame comprising a plurality of interconnected struts; a plurality of leaflets arranged on an inside of the frame, each leaflet of the plurality of leaflets comprising two commissure tabs disposed on opposite sides of the leaflet and a cusp edge portion extending between the two commissure tabs, wherein the cusp edge portions of the plurality of leaflets are attached directly to a plurality of first struts of the plurality of interconnected struts, and wherein the plurality of first struts have a curved shape configured to follow a curvature of the cusp edge portions of the plurality of leaflets; and an outer skirt disposed around an outer surface of the frame.

Example 55. The prosthetic heart valve of any example herein, particularly example 54, wherein the plurality of first struts are arranged end-to-end with one another and undulate around a circumference of the frame between commissure windows of the frame and an end of the frame, wherein the commissure windows are configured to receive the commissure tabs of the plurality of leaflets.

Example 56. The prosthetic heart valve of any example herein, particularly example 54 or example 55, wherein the plurality of first struts include a plurality of inflow support struts spaced apart from one another around a circumference of the frame, each inflow support strut of the plurality of inflow support struts configured to receive and follow a curvature of an inflow end of a cusp edge portion of a corresponding leaflet of the plurality of leaflets and having a first radius of curvature that is greater than a second radius of curvature of a remaining portion of the plurality of first struts.

Example 57. The prosthetic heart valve of any example herein, particularly example 56, wherein the plurality of inflow support struts is configured to have increased flexibility relative to remaining struts of the plurality of interconnected struts of the frame and bend along at least a portion of each inflow support strut of the plurality of inflow support struts.

Example 58. The prosthetic heart valve of any example herein, particularly any one of examples 54-57, wherein the plurality of interconnected struts further comprises a plurality of second struts, and wherein each second strut of the plurality of second struts is angled and straight along a majority of its length.

Example 59. The prosthetic heart valve of any example herein, particularly any one of examples 54-58, wherein the outer skirt is directly attached to the plurality of interconnected struts and extends between a first end of the frame and a location between the first end and a second end of the frame.

Example 60. The prosthetic heart valve of any example herein, particularly any one of examples 54-59, wherein the outer skirt is attached the plurality of interconnected struts by a plurality of loose stitches configured to allow the outer skirt to float along the plurality of interconnected struts without stretching in a longitudinal direction during radial compression of the frame.

Example 61. The prosthetic heart valve of any example herein, particularly any one of examples 54-60, wherein edges of the outer skirt that are disposed at opposite ends of the outer skirt are folded over, against an outer surface of the outer skirt that faces away from the frame, to form a folded over first end and a folded over second end of the outer skirt.

Example 62. The prosthetic heart valve of any example herein, particularly example 61, wherein the folded over first end and the folded over second end of the outer skirt are each folded over a monofilament.

Example 63. The prosthetic heart valve of any example herein, particularly example 62, wherein the monofilament is radiopaque.

Example 64. The prosthetic heart valve of any example herein, particularly any one of examples 54-63, wherein the outer skirt comprises ePTFE.

Example 65. The prosthetic heart valve of any example herein, particularly any one of examples 54-64, wherein the outer skirt includes a first end and a second end arranged opposite one another and wherein one or more of the first end and the second end undulate around a circumference of the frame.

In view of the many possible examples to which the principles of the disclosed technology may be applied, it should be recognized that the illustrated examples are only preferred examples of the disclosed technology and should not be taken as limiting the scope of the claimed subject matter. Rather, the scope of the claimed subject matter is defined by the following claims and their equivalents.

We claim:

1. A prosthetic heart valve comprising:
a radially expandable and compressible annular frame comprising:
a plurality of interconnected struts defining a plurality of rows of cells arranged between a first end and a second end of the frame, the plurality of interconnected struts comprising a plurality of first struts defining the first end of the frame; and
a plurality of second struts axially offset from the first end of the frame, each second strut connected to and between two adjacent first struts of the plurality of first struts and extending axially away from the two adjacent first struts; and
a plurality of leaflets attached to the frame, wherein each leaflet of the plurality of leaflets comprises a cusp edge portion and a free edge portion, and wherein an inflow end of the cusp edge portion of each leaflet of the plurality of leaflets is secured to a corresponding second strut of the plurality of second struts such that the inflow end is axially offset from the first end of the frame.

2. The prosthetic heart valve of claim 1, wherein the plurality of interconnected struts is configured as structural struts of the frame that support a radial expansion and compression of the frame and provide structure to the frame in a radially expanded configuration, and wherein the plurality of second struts is configured to be non-structural to the frame and provide support to inflow ends of the plurality of leaflets.

3. The prosthetic heart valve of claim 1, wherein a total number of second struts of the plurality of second struts equals a total number of leaflets of the plurality of leaflets, and wherein the plurality of second struts is spaced apart from one another around a circumference of the frame.

4. The prosthetic heart valve of claim 1, wherein each second strut of the plurality of second struts curves from a first end of the second strut to a second end of the second strut, the first end connected to a first junction between a first pair of first struts of the plurality of first struts and the second end connected to a second junction between a second pair of first struts of the plurality of first struts.

5. The prosthetic heart valve of claim 4, wherein each second strut has a radius of curvature that follows a radius of curvature of the inflow end of the cusp edge portion of a corresponding leaflet, and wherein each second strut curves by a greater amount than each first strut of the plurality of first struts.

6. The prosthetic heart valve of claim 1, wherein the plurality of second struts has a second width that is smaller than a first width of the plurality of first struts.

7. The prosthetic heart valve of claim 1, wherein each second strut of the plurality of second struts has one or more thinned regions which has a width that is smaller than a width of a remainder of the second strut and a width of the plurality of first struts.

8. The prosthetic heart valve of claim 1, wherein, for each leaflet, the inflow end of the cusp edge portion of the leaflet is directly attached to the corresponding second strut such that the inflow end of the cusp edge portion of the leaflet is offset from the first end of the frame and an outflow edge of the leaflet is offset from the second end of the frame, toward the first end of the frame, and wherein the first end of the frame is an inflow end of the frame and the second end of the frame is an outflow end of the frame.

9. The prosthetic heart valve of claim 1, wherein, for each leaflet, the inflow end of the cusp edge portion of the leaflet is secured directly to the corresponding second strut by one or more sutures, further comprising an outer skirt disposed around an outer surface of the frame, and wherein the one or more sutures attach both the inflow ends of the cusp edge portions of the leaflets and an inflow end of the outer skirt to the frame, the plurality of leaflets arranged on an inside of the frame.

10. The prosthetic heart valve of claim 1, wherein the plurality of first struts includes a plurality of cusp edge portion support struts that extend from a corresponding second strut toward a commissure window of the frame, wherein the plurality of first struts includes a plurality of angled linear struts, wherein the plurality of cusp edge portion support struts has a greater curvature than the plurality of angled linear struts, and wherein the greater curvature of the plurality of cusp edge portion support struts is configured to follow a curvature of the cusp edge portion of the plurality of leaflets.

11. A prosthetic heart valve comprising:
a radially expandable and compressible annular frame comprising:
a plurality of interconnected struts defining a plurality of rows of cells arranged between an inflow end and an outflow end of the frame, the plurality of interconnected struts comprising:
a plurality of first struts defining a plurality of commissure windows disposed adjacent to the outflow end of the frame; and
a plurality of second struts arranged end-to-end and extending between the plurality of commissure windows and the inflow end of the frame in an undulating pattern around a circumference of the frame; and
a plurality of third struts axially offset from the inflow end of the frame, each third strut of the plurality of third struts connected to two adjacent second struts of the plurality of second struts which form a part of the inflow end of the frame.

12. The prosthetic heart valve of claim 11, further comprising a plurality of leaflets attached directly to the frame, wherein each leaflet comprises two commissure tabs disposed on opposite sides of the leaflet and a cusp edge portion extending between the two commissure tabs, and wherein the cusp edge portion of each leaflet is secured directly to the plurality of second struts and the plurality of third struts.

13. The prosthetic heart valve of claim 12, wherein an inflow end of the cusp edge portion of each leaflet is secured directly to a corresponding third strut of the plurality of third struts and wherein each third strut of the plurality of third struts is curved between its ends such that each third strut follows a curvature of the inflow end of the cusp edge portion of the leaflet secured thereto.

14. The prosthetic heart valve of claim 13, wherein each second strut of the plurality of second struts is curved to follow a curvature of a portion the cusp edge portion of the leaflet secured directly thereto, and wherein a first radius of curvature of the plurality of third struts is greater than a second radius of curvature of the plurality of second struts.

15. The prosthetic heart valve of claim 11, further comprising an outer skirt disposed around an outer surface of the frame, the outer skirt having an inflow edge and outflow edge that are folded over and disposed against an outer surface of the outer skirt, and wherein the plurality of leaflets and the outer skirt are secured to the plurality of second struts and the plurality of third struts by a plurality of loose stitches.

16. The prosthetic heart valve of claim 11, wherein the plurality of interconnected struts further comprises a plurality of fourth struts that are angled and straight along a majority of their length, and wherein each third strut is further connected to two adjacent fourth struts forming a part of the inflow end of the frame.

17. A prosthetic heart valve comprising:
a radially expandable and compressible annular frame comprising:
a plurality of interconnected struts defining a plurality of rows of cells arranged between an inflow end and an outflow end of the frame and configured to support a radial expansion and compression of the frame, the plurality of interconnected struts including a plurality of first struts arranged end-to-end around a circumference of the frame and forming the inflow end of the frame;
a plurality of apices formed at the inflow end of the frame, each apex of the plurality of apices formed at a junction between two adjacent first struts of the plurality of first struts; and
a plurality of arcuate second struts that extend axially beyond the inflow end of the frame, where each second strut of the plurality of second struts is connected to and between two adjacent apices of the plurality of apices and is configured to bend at a central region of the second strut during radial compression of the frame.

18. The prosthetic heart valve of claim 17, wherein each second strut of the plurality of second struts comprises one or more regions that have a first width that is smaller than a second width of the plurality of first struts.

19. The prosthetic heart valve of claim 17, further comprising a plurality of leaflets, each leaflet comprising a cusp edge portion extending between two commissure tabs of the leaflet, wherein an inflow end of the cusp edge portion of each leaflet is secured directly to a corresponding second strut of the plurality of second struts, wherein a remainder of the cusp edge portion of each leaflet is secured directly to a first portion of the plurality of first struts that extend from commissure windows of the frame to the plurality of second struts, and wherein the commissure windows are configured to receive commissure tabs of adjacent leaflets of the plurality of leaflets.

20. The prosthetic heart valve of claim 19, wherein each first strut of the first portion of the plurality of first struts is curved and configured to follow a shape of a portion of the cusp edge portion of the leaflet secured thereto.
